(19) United States
(12) Patent Application Publication
(10) Pub. No.: US 2021/0023384 A1
(43) Pub. Date: Jan. 28, 2021

(54) NON-INVASIVE CORNEA AND SCLERA STRENGTHENING DEVICE AND METHOD

(71) Applicant: TSUBOTA LABORATORY, INC., Tokyo (JP)

(72) Inventors: Hidenaga KOBASHI, Tokyo (JP); Kazuo TSUBOTA, Tokyo (JP)

(73) Assignee: TSUBOTA LABORATORY, INC., Tokyo (JP)

(21) Appl. No.: 16/982,152

(22) PCT Filed: Mar. 19, 2019
(86) PCT No.: PCT/JP2019/011552

§ 371 Date: Sep. 18, 2020

(30) Foreign Application Priority Data
Mar. 21, 2018 (JP) 2018-053647

(51) Int. Cl. A61N 5/06; A61F 9/00

(52) U.S. Cl. CPC A61N 5/062; A61F 9/0026; A61N 2005/0663; A61N 2005/0651; A61N 2005/0648

(57) ABSTRACT

To provide a device and a method for strengthening a cornea or a sclera or suppressing a progression of a disease in a non-invasive manner, enabling treatment in daily life without treatment in a confined state at a medical institution for a fixed period of time and without ablation of the corneal epithelium as in conventional corneal crosslinking. The above-described problem is solved by a non-invasive cornea and sclera strengthening device that irradiates violet light toward an eye administered with an administration agent used to strengthen a corneal tissue or a scleral tissue, and is configured so that an irradiance of the violet light onto a surface of the eye is within a range of 0.1 to 1 mW/cm², and a time of irradiation of the violet light onto the eye is within a range of one to five hours per day.

### FIELD OF THE INVENTION

The present invention relates to a non-invasive cornea and sclera strengthening device and method, and more specifically relates to a device and a method for strengthening a cornea or a sclera or suppressing a progression of a disease in a non-invasive manner, enabling treatment in daily life without receiving conventional corneal crosslinking, which is a treatment for suppressing the progression of keratoconus and other corneal diseases, at a medical institution, and without ablating a corneal epithelium.

### BACKGROUND ART

Keratoconus is a progressive disease in which a thickness near a center of a cornea becomes thin and the cornea projects conically forward. Conventionally, corneal crosslinking (abbreviated as “CXL”) has been proposed as a treatment method for suppressing the progression of such keratoconus and other corneal diseases (for example, ectasia after keratorefractive surgery, pellucid marginal corneal degeneration, and the like), and has been implemented on the basis of high evidence. However, this CXL normally requires surgical ablation of the corneal epithelium in order to penetrate a corneal parenchyma with riboflavin ophthalmic solution. The surgery is highly invasive and may lead to postoperative pain and cornea-infectious complications. Furthermore, in a normal CXL, the patient has the inconvenience of being subjected to light irradiation treatment in a confined state in an operating room of a medical institution, and an irradiance of the light irradiation is also high and thus eye complications due to ultraviolet A (UVA) are a concern as well.

With regard to conventional CXL, in Non-Patent Documents 1 and 2, it is reported that photosensitizing riboflavin is applied to a human or the like, and UVA (wavelength: 370 nm, irradiance: 3 mW/cm²) is irradiated onto the eye for 30 minutes at a distance of 1 cm. Further, in Non-Patent Document 3, it is reported that photosensitizing riboflavin is applied to a rabbit, and UVA (wavelength: 370 nm, irradiance: 3 mW/cm²) is irradiated onto the eye for 30 minutes at a distance of 1 cm. Furthermore, in Non-Patent Document 4, it is reported that photosensitizing riboflavin is applied to a guinea pig, and UVA (wavelength: 370 nm, irradiance: 57 mW/cm²) is irradiated onto the eye for a total of 20 minutes at a fixed distance of 2±3 cm.

### PRIOR ART DOCUMENTS

### Non-Patent Documents

Non-Patent Document 1: Wollensak G, Spoerl E, Seiler T., Am. J. Ophthalmol., May 2003; 135 (5): 620-7 Non-Patent Document 2: Gregor Wollensak, Eberhard Spoerl, Theo Seiler, J. Cataract. Refract. Surg-Vol. 29, September 2003. 1780-1785 Non-Patent Document 3: Gregor Wollensak, Elena Iomdina, Dag-Daniel Dittert, Olga Salamatina and Gisela Stoltenburg, Acta Ophthalmol. Scand. 2005: 83: 477-482 Non-Patent Document 4: Shuai Liu 1, Shengjie Li 1, Bingjie Wang 1, Xiao Lin 1, Yi Wu, Hong Liu 1, Xiaomei Qu, Jinhui Dai, Xingtao Zhou, Hao Zhou, Plos One, Nov. 9, 2016, 1-16

### SUMMARY OF THE INVENTION

### Problems to be Solved by the Invention

The present invention solves the problems of conventional invasive corneal crosslinking (also abbreviated as CXL) in which the corneal epithelium is ablated and UVA is irradiated at a high irradiance, and an object thereof is to provide a device (referred to as “non-invasive cornea and sclera strengthening device” in the present application) and a method for strengthening a cornea or a sclera or suppressing a progression of a disease in a non-invasive manner, enabling treatment in daily life without treatment in a confined state at a medical institution for a fixed period of time, and without ablation of the corneal epithelium as in conventional CXL.

### Means for Solving the Problems

(1) A non-invasive cornea and sclera strengthening device according to the present invention is a device that irradiates violet light toward an eye administered with an administration agent used to strengthen a corneal or a scleral tissue, an irradiance of the violet light onto a surface of the eye is within a range of 0.1 to 1 mW/cm², and a time of irradiation of the violet light onto the eye is within a range of one to five hours per day.

According to this invention, by the device that irradiates the violet light toward the eye administered with an administration agent used for strengthening the tissue of the cornea or the sclera for a predetermined period of time at a low irradiance within the above-described range, a new treatment technique that realizes the same effect as that of conventional corneal crosslinking (suppression of the progression of keratoconus, other corneal diseases, and the like) was discovered. This treatment technique can realize the same effect not only on the corneal tissue but also on the scleral tissue and, because irradiation is performed at a low irradiance, the impact on the eye can be eliminated or minimized. Furthermore, it is possible to utilize a violet light source having a low irradiance, and thus make a portable irradiating device such as eyeglasses, and carry out treatment in daily life without treatment in a confined state at a medical institution for a fixed period of time.

Preferably the non-invasive cornea and sclera strengthening device according to the present invention is a facially oriented-type device disposed so that the violet light is irradiated in a direction of the eye, and is one or two or more devices selected from facially oriented-type light source devices, such as eyeglasses with a light source, a liquid crystal display with a light source, a desk lamp, a handy light source, a table-top installed-type light source, and a personal computer/mobile terminal mounted-type light source.

According to this invention, because the device is a facially oriented-type device disposed in front or forward of the face, irradiating the violet light in the direction of the eye, irradiation can be performed toward the eye at the irradiance described above for a predetermined period of time only. In particular, by configuring the device so that the violet light is emitted from various devices used in daily life, it is possible to realize treatment in daily life.

In the non-invasive cornea and sclera strengthening device according to the present invention, preferably an administrating device of the administration agent is integrated with the facially oriented-type device.

According to this invention, because the administrating device of the administration agent is integrated with the facially oriented-type device, the device is compact and portable, and treatment can be carried out without causing the subject to feel any burden.

In the non-invasive cornea and sclera strengthening device according to the present invention, preferably the administration agent is administered to the eye by one or two or more methods selected from eye drops, eye ointment, sustained release from an eyeglass frame, and sustained release from a contact lens.

According to this invention, the administration agent can be easily administered. Furthermore, by wearing a contact lens that transmits violet light, the progression of keratoconus and other corneal diseases (for example, corneal ectasia and the like) can be delayed or suppressed.

In the non-invasive cornea and sclera strengthening device according to the present invention, the contact lens transmits violet light. It should be noted that a ratio (transmittance) of violet light transmitted through the contact lens is preferably 60 to 100%.

In the non-invasive cornea and sclera strengthening device according to the present invention, preferably administration of the administration agent is carried out periodically or continuously.

In the non-invasive cornea and sclera strengthening device according to the present invention, preferably the non-invasive cornea and sclera strengthening device is applied to keratoconus or other corneal diseases such as corneal ectasia, corneal infection, bullous keratopathy or the like, and ametropia.

(2) A non-invasive cornea and sclera strengthening device according to the present invention is a device that irradiates violet light toward an eye to strengthen a tissue of a cornea or a sclera, an irradiance of the violet light onto a surface of the eye is within a range of 0.1 to 1 mW/cm², and a time of irradiation of the violet light onto the eye is within a range of one to five hours per day.

Preferably the non-invasive cornea and sclera strengthening device according to the present invention is one or two or more of the following: (a) the non-invasive cornea and sclera strengthening device is a facially oriented-type device disposed so that the violet light is irradiated in a direction of the eye, and is one or two or more devices selected from facially oriented-type light source devices, such as eyeglasses with a light source, a liquid crystal display with a light source, a desk lamp, a handy light source, a table-top installed-type light source, and a personal computer/mobile terminal mounted-type light source, (b) the contact lens transmits violet light, and (c) the non-invasive cornea and sclera strengthening device is applied to keratoconus or other corneal diseases such as corneal ectasia, corneal infection, bullous keratopathy or the like, and ametropia.

(3) A non-invasive cornea and sclera strengthening method according to the present invention comprises the step of using a non-invasive cornea and sclera strengthening device capable of setting an irradiance of violet light onto a surface of an eye within a range of 0.1 to 1 mW/cm², and setting a time of irradiation of the violet light onto the eye within a range of one to five hours per day and, at a timing after administration of an administration agent used for strengthening a tissue of a cornea or sclera to the eye, irradiating the violet light by the non-invasive cornea and sclera strengthening device.

(4) A non-invasive cornea and sclera strengthening method according to the present invention comprises the step of using a non-invasive cornea and sclera strengthening device capable of setting an irradiance of violet light onto a surface of an eye within a range of 0.1 to 1 mW/cm², and setting a time of irradiation of the violet light onto the eye within a range of one to five hours per day.

### Effect of the Invention

According to the present invention, it is possible to provide a non-invasive cornea and sclera strengthening device and method that enable treatment in daily life without treatment in a confined state at a medical institution for a fixed period of time and without ablation of the corneal epithelium as in conventional corneal crosslinking.

### BRIEF DESCRIPTION OF THE DRAWINGS

FIG. 1 is a graph showing tensile test results of a corneal tissue.

FIG. 2 is a graph showing tensile test results of a scleral tissue.

FIG. 3 is a graph showing Young's modulus of the corneal tissue and the scleral tissue.

FIG. 4 is a conceptual view of the present invention and a conventional method.

FIGS. 5A and 5B are images showing examples of eyeglasses integrated with an administration agent mist spraying device.

FIG. 6 is a graph showing the sustained-release amount from a riboflavin-containing soft contact lens.

FIG. 7 is a graph showing tensile test results in a case of implementation on a pig eye cornea.

FIG. 8 is a graph showing change over time of a maximum value of a refractive power (D) of a human cornea.

FIG. 9 is a graph showing change over time of best spectacle-corrected visual acuity (BSCVA).

FIG. 10 is a graph showing tensile test results in a case of implementation on a pig eye cornea.

### EMBODIMENTS OF THE INVENTION

A non-invasive cornea and sclera strengthening device and method according to the present invention are described below with reference to the drawings. The present invention is not limited to the contents of the following embodiments and examples, and includes various modifications and applications within the scope of the gist of the present invention.

### Non-Invasive Cornea and Sclera Strengthening Device

A non-invasive cornea and sclera strengthening device according to the present invention is a device that irradiates violet light toward an eye administered with an administration agent used to strengthen a corneal tissue or a scleral tissue, an irradiance of the violet light onto a surface of the eye is within a range of 0.1 to 1 mW/cm², and a time of irradiation of the violet light onto the eye is within a range of one to five hours per day.

With this non-invasive cornea and sclera strengthening device, by a device that irradiates the violet light toward the eye administered with an administration agent used for strengthening the corneal tissue or the scleral tissue for a predetermined period of time at a low irradiance within the above-described range, a new treatment technique that realizes the same effect as that of conventional corneal crosslinking (suppression of the progression of keratoconus, other corneal diseases, and the like) was discovered. This treatment technique can realize the same effect not only on the corneal tissue but also on the scleral tissue and, because irradiation is performed at a low irradiance, the impact on the eye can be eliminated or minimized. Furthermore, it is possible to utilize a violet light source having a low irradiance, and thus make a portable irradiating device such as eyeglasses, and carry out treatment in daily life without treatment in a confined state at a medical institution for a fixed period of time.

Furthermore, in subsequent research, it was revealed that the same effect as that of conventional corneal crosslinking (suppression of progression of keratoconus, other corneal diseases, and the like) can be realized by simply irradiating violet light at a low irradiance within the above-described range for a predetermined period of time.

Hereinafter, each component will be described in detail. It should be noted that the present inventors may call this new technology “KeraVio.”

### Administration Agent/Photo-Crosslinking Agent

The administration agent is an agent administered to the eye, and is a photo-crosslinking agent used for strengthening the corneal tissue or the scleral tissue by irradiation with light after administration. This administration agent has characteristics of crosslinking collagen fibers constituting the cornea and the sclera and strengthening (crosslinking and strengthening) a collagen tissue by a wavelength and an irradiance of violet light described later. As long as the administration agent has these characteristics, the type thereof may be one currently known or developed in the future, and is not particularly limited. Examples of such an administration agent include an agent containing flavin adenine dinucleotide (FAD), which is a coenzyme-type vitamin B2, as a main component, verteporfin, indocyanine green (ICG), cyanocobalamin (a typical cobalamin, also called vitamin B12), and the like, but are not limited thereto.

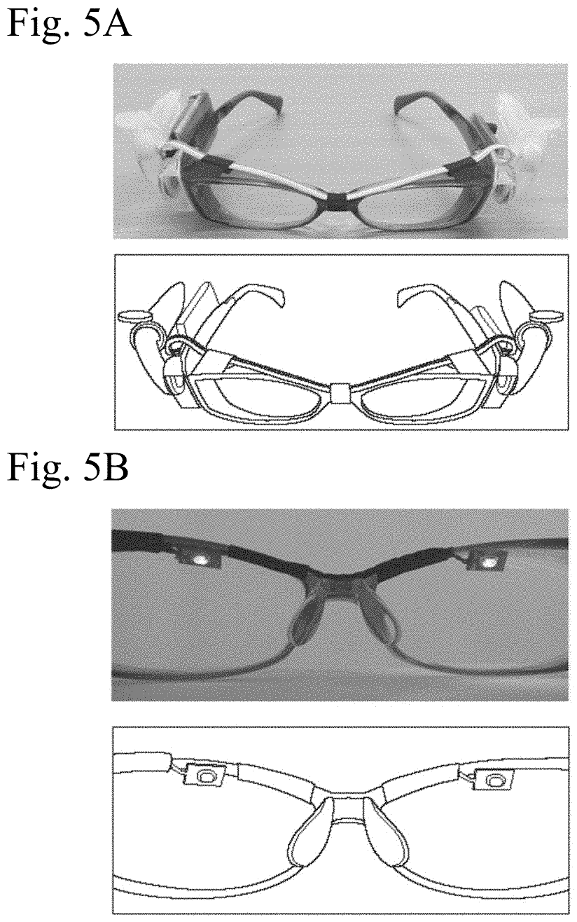

The means of administration may be various methods and, while not particularly limited, examples thereof include eye drops, eye ointment, sustained release from eyeglasses having a sustained-release function, sustained release from a contact lens having a sustained-release function, and the like. By these release means, the administration agent can be easily administered to the eye. The eye drops and the eye ointment are normal means for administering the administration agent directly to the eye. The sustained release from eyeglasses having a sustained-release function is a means for, for example, administering an administration agent formed into a mist spray, from a mist spraying device provided to the eyeglass frame, for example, as in the eyeglasses integrated with an administration agent mist spraying device shown in FIGS. 5A and 5B. The sustained release from a contact lens having a sustained-release function is a means performed by wearing a contact lens impregnated with an administration agent onto the eye, thereby causing the administration agent to be gradually administered from the contact lens to the eye. The contact lens is a sustained-release contact lens that transmits violet light described later, and by applying and wearing such a contact lens, it is possible to effectively delay or suppress the progression of keratoconus and other corneal diseases (for example, corneal ectasia and the like).

Among these, the eyeglasses having a sustained-release function and the contact lens having a sustained-release function each function as an administrating device of an administration agent, and can be said to be a facially oriented-type integrated device in a broad sense or a narrow sense. With such an integrated device, the device is compact and portable, and treatment can be carried out without causing the subject to feel any burden. It should be noted that in the contact lens having a sustained-release function, a ratio (transmittance) of violet light (all or part of wavelength light within a range of 360 to 400 nm) transmitted through the contact lens is preferably 60 to 100%. The term “all” refers to all within the range of 360 to 400 nm, and the term “part” refers to wavelengths within that range, such as 365 nm and 375 nm, for example.

The administration of the administration agent is preferably carried out periodically or continuously. The eye drops and eye ointment are periodically applied at regular intervals or irregular intervals. The eyeglasses having a sustained-release function control a mist spraying device mounted to an eyeglass frame, for example, making it possible to periodically or continuously carry out administration at regular intervals or irregular intervals. The contact lens having a sustained-release function allows continual administration due to the configuration thereof. It should be noted that the “regular intervals and irregular intervals” are also not particularly limited, and may be selected as desired taking into consideration factors such as a required intensity level, a lifestyle pattern, administration means, a type of administration agent, an irradiation intensity of violet light described later, and an irradiation time.

It is desirable that the administration time is long to the extent that the daily lifestyle pattern is not hindered. In the present invention, treatment is not carried out in a confined state in a medical institution such as a hospital, but is carried out in daily life, and thus may be carried out within normal living hours. A time that does not feel too burdensome is, for example, about one to five hours. In the case of continuous sustained release, for eyeglasses with a sustained-release function, the eyeglasses may be worn during that time, and the release may be carried out by adjusting the mist spray amount within the wearing time, and for a sustained-release contact lens as well, the lens may be worn during that time, and the release may be carried out within the wearing time. In the case of intermittent administration (at regular intervals or irregular intervals), for eye drops and eye ointment, the eye drops or eye ointment may be administered every 30 minutes, or the like, for example, and for eyeglasses with a sustained-release function, the mist may be sprayed for every fixed period of time by intermittent control, or the like.

The administration amount and content of the administration agent are also not particularly limited, and may be selected as desired taking into consideration factors such as the required intensity level, the lifestyle pattern, the administration means, the type of administration agent, the irradiation intensity of violet light described later, and the irradiation time. The administration agent is normally an aqueous solution, and the content is the content included in the aqueous solution. It should be noted that, as an example, the content of vitamin B2 in the ophthalmic solution and the eye ointment may be the same or substantially the same as that of a commercial product available on the market, and, for example, 0.05% to 0.1%.

A preservative such as benzalkonium chloride may be blended into the administration agent within a range that does not impair the effects of the present invention. The blending amount may also be within a range that does not impair the effects of the present invention, and, for example, 0.005 to 0.02 mass % or less.

### Violet Light

Violet light, depending on a wavelength and an irradiance thereof, causes a photoreaction in the administration agent described above, and acts to crosslink the collagen fibers constituting the cornea and the sclera and strengthen the collagen tissue. The violet light can be said to have all or part of wavelengths within the range of 360 to 400 nm, but a preferable wavelength is selected in relation to the administration agent. The violet light may have all of these wavelengths, or may have a specific peak wavelength within the range, and the wavelength is not particularly limited. Particularly, a wavelength of 370 nm is preferable, but violet light having a peak within a range of 365 to 380 nm can be preferably used. The present inventors found that, even when this violet light is irradiated onto the eye at a considerably weak irradiance (for example, about 1/10 of the irradiance in the related art), the strength of the cornea or the sclera can be increased by using the violet light together with a predetermined administration agent.

The irradiance of the violet light is preferably within a range of 0.1 to 1 mW/cm². It is possible to perform the irradiation at such a low irradiance, and thus make a portable irradiating device such as eyeglasses, and carry out treatment in daily life without treatment in a confined state at a medical institution for a fixed period of time. The irradiance is related to the photo-crosslinking effect of the administration agent, and thus the violet light is applied in combination with an administration agent in which a crosslinking reaction occurs at such a low irradiance as well. In the present invention, because treatment is carried out at a low irradiance, the photo-crosslinking reaction can conceivably be slowly carried out, the collagen tissue is strengthened as a result, and thus the collagen fibers can conceivably be favorably crosslinked. By such a technique, the same effect as or a greater effect than that of conventional corneal crosslinking (suppression of progression of keratoconus, other corneal diseases, and the like) can be realized. This technique can realize the same effect not only on the corneal tissue but also on the scleral tissue and, because irradiation is performed at a low irradiance, the impact on the eye can be eliminated or minimized.

Furthermore, in subsequent research, it was revealed that the same effect as that of conventional corneal crosslinking (suppression of progression of keratoconus, other corneal diseases, and the like) can be realized as in experimental examples described later by simply irradiating violet light at a low irradiance within the above-described range for a predetermined period of time.

When the irradiation intensity is less than 0.1 mW/cm², the photo-crosslinking reaction may not sufficiently occur, and conceivably irradiation for a long period of time may be required to strengthen the cornea or the sclera. On the other hand, when the irradiation intensity exceeds 1 mW/cm², the intensity is somewhat strong, although weaker than in the related art, and thus this value is preferably set as the upper limit taking into consideration that a portable light source may not be readily available, and the like.

The irradiation time of the violet light is preferably within a range of one to five hours per day. Such light can be made intermittent (regular intervals or irregular intervals) or continuous as desired. One to five hours per day is a length of time that does not interfere with a daily lifestyle pattern, as explained in the description of the administration agent as well. In the present invention, treatment is not carried out in a confined state in a medical institution such as a hospital, but is carried out in daily life, and thus may be carried out, for example, for about one to five hours within normal living hours, which does not feel too burdensome.

Preferable examples of the irradiating device of the violet light include a facially oriented-type device disposed so that the violet light is irradiated in the direction of the eye. As examples, preferably the irradiating device is one or two or more devices selected from facially oriented-type light source devices, such as eyeglasses with a light source, a liquid crystal display with a light source, a desk lamp, a handy light source, a table-top installed-type light source, and a personal computer/mobile terminal mounted-type light source. Such facially oriented-type devices can irradiate toward the eye at the irradiance described above for a predetermined period of time only, and thus, in particular, by configuring the device so that the violet light is emitted from various devices used in daily life, treatment in daily life can be realized.

### Other

The non-invasive cornea and scleral strengthening device can be provided with various functional parts and members. For example, a notification device may be provided for notifying that the administration agent is to be administered by eye drops or eye ointment for every fixed period of time. This notification can be made via a smartphone or a tablet terminal application.

Further, a method using such a non-invasive cornea and sclera strengthening device includes the step of using the non-invasive cornea and sclera strengthening device capable of setting an irradiance of violet light onto a surface of the eye within a range of 0.1 to 1 mW/cm², and setting a time of irradiation of the violet light onto the eye within a range of one to five hours per day, and is capable of realizing, at an activation timing after administration of an administration agent used for strengthening a corneal tissue or a scleral tissue to the eye, irradiation of the violet light by the non-invasive cornea and sclera strengthening device. Preferably such activation timing is set in advance by a computer as desired, and the device is configured so that the timing can be input as desired. Examples of input items include violet light irradiance, intermittent (regular intervals or irregular intervals) or continuous irradiation, irradiation time, and the like. Furthermore, when a mist spraying function is provided, examples include administration time, administration timing (intermittent (regular intervals or irregular intervals) or continuous), administration amount, and the like.

Further, as described above, in subsequent research, it was revealed that the same effect as that of conventional corneal crosslinking (suppression of progression of keratoconus, other corneal diseases, and the like) can be realized as in the experimental examples described later by simply irradiating violet light at a low irradiance within the above-described range for a predetermined period of time.

As described above, the non-invasive cornea and sclera strengthening device according to the present invention enables treatment in daily life without treatment in a confined state at a medical institution for a fixed period of time and without ablation of the corneal epithelium as in conventional corneal crosslinking. As for targeted eye diseases, the device can be applied to keratoconus and other corneal diseases (corneal ectasia, corneal infection, bullous keratopathy, bullous keratopathy, ametropia, and the like).

### EXAMPLES

The present invention is described in further detail below using experimental examples.

### Experiment 1

With a rabbit eyeball targeted, flavin adenine dinucleotide (FAD) ophthalmic solution containing 0.53 mg/mL of FAD was used to measure changes in strength in corneal and scleral tissues due to violet light irradiation. As the violet light, a light-emitting diode (LED) having a wavelength of 375 nm (manufactured by Nitride Semiconductors Co., Ltd., model name: Flashlight) was used, and the irradiance thereof was set to 310 μW/cm². This violet light was continuously irradiated for three hours per day, and eye drops were applied six times total at intervals of 30 minutes during the three hours. The total eye drop amount was 50 to 60 μg.

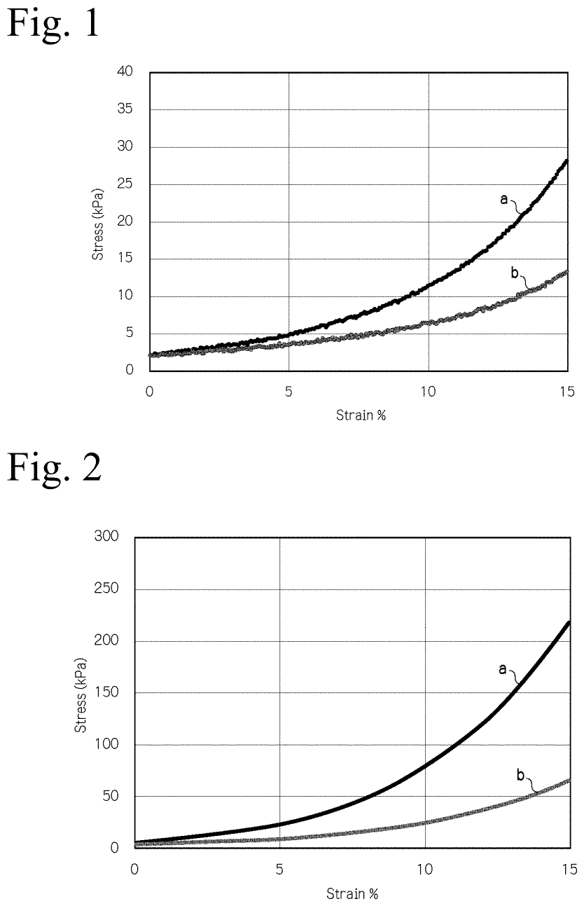

This was performed for one week (seven days). In all examples, epithelial ablation was not performed. After one week elapsed, the corneal and scleral tissues of the rabbit were collected. A corneal/scleral tissue strip having a width of 5 mm and a length of 10 mm was prepared, and a portion of a width of 5 mm and a length of 5 mm was fixed to a tensile tester (TA. XTplus; Stable Micro Systems, U.K.). The tensile strength was measured by pulling in the longitudinal direction at a speed of 0.029 mm/sec. FIG. 1 shows the results of the tensile test of the corneal tissue, and FIG. 2 shows the results of the tensile test of the scleral tissue. In both FIG. 1 and FIG. 2, the reference sign a denotes the result of the present invention, and the reference sign b denotes the result of the control. It should be noted that corneal and scleral tissues to which eye drops and irradiation were not applied were used as the controls (reference sign b in FIG. 1 and reference sign b in FIG. 2).

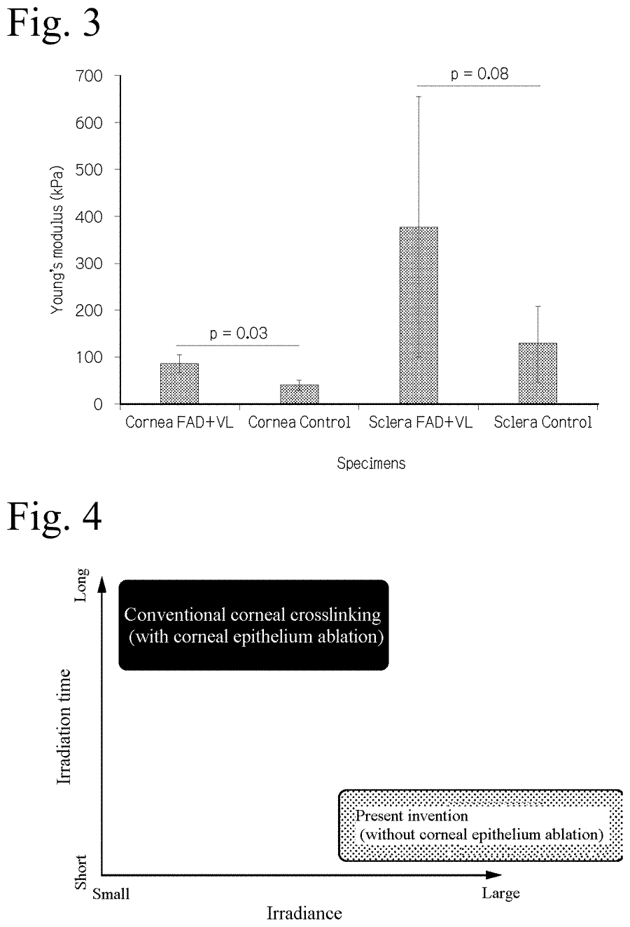

Increases in the strength of the corneal and scleral tissues by the FAD eye drops and violet light irradiation were confirmed. Further, as shown in FIG. 3, Young's modulus increased after the FAD eye drops and violet light irradiation. In FIG. 3, “Cornea” refers to the cornea and “Sclera” refers to the sclera. Furthermore, changes in the cornea and crystalline lens were not found before and after the experiment using a slit lamp microscope.

### Evaluation

The violet light irradiance of the present invention is 310 μW/cm², which is approximately 10% of the 3.0 mW/cm² of the conventional method, making it possible to avoid adverse events and side effects associated with UVA. FIG. 4 is a conceptual view of the corneal crosslinking of the conventional method and the non-invasive corneal and scleral strengthening device of the present invention. It was found that, by irradiating at a low irradiance for a long time, the present invention has the same effect as that of the conventional method. While the conventional method involves corneal epithelium ablation, is performed at a high irradiance, and is therefore relatively highly invasive, the present invention allows irradiation from a facially oriented-type device, making it possible to perform self-treatment without treatment in a confined state in a medical institution. That is, the patient can practice treatment in his or her daily life and many patients can be given the opportunity to utilize the device.

Further, in the conventional method, it is necessary to ablate the corneal epithelium, which is invasive, but in the present invention, treatment can be performed without ablation, resulting in the advantage that complications such as postoperative pain and corneal infections can be avoided. Further, the present invention is minimally invasive, and therefore can be adapted to children, atopic dermatitis, Down's syndrome, and severe dry eye as well, which were careful adaptations in the conventional method, and can expand a range thereof. Further, because a hard contact lens (HCL) is worn to correct refraction in most keratoconus cases, it is inconvenient that the conventional method prohibits the wearing of a HCL immediately after surgery. The present invention, however, allows the wearing of a HCL during treatment as well. Furthermore, when the sustained-release eyeglasses or the sustained-release contact lenses are used, the time and effort of eye drops for a fixed period of time can be omitted.

### Experiment 2

In Experiment 2, an actual device was applied. FIGS. 5A and 5B are images showing examples of eyeglasses integrated with an administration agent mist spraying device. In these eyeglasses integrated with an administration agent mist spraying device, as shown in FIG. 5B, the mist is sprayed from the administration agent mist spraying device mounted to the frame portion (temple portion) toward the eye to administer the administration agent mist. Further, as shown in FIG. 5A, a light source of the violet light is mounted to upper frame portions on both sides.

### Experiment 3

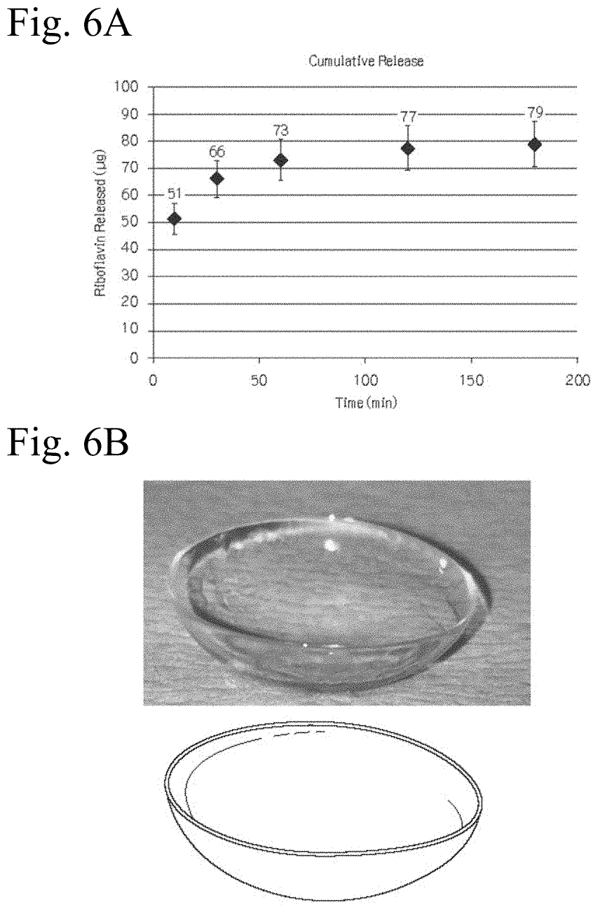

In Experiment 3, a riboflavin-containing soft contact lens was used. FIG. 6A is a graph showing the sustained-release amount from the riboflavin-containing soft contact lenses. It is understood that, by using a contact lens having such a sustained-release performance, the present invention can be effectively carried out. It should be noted that, from FIG. 6A, it is understood that the FAD component is gradually released over time. FIG. 6B is an image of the riboflavin-containing soft contact lens.

### Experiment 4

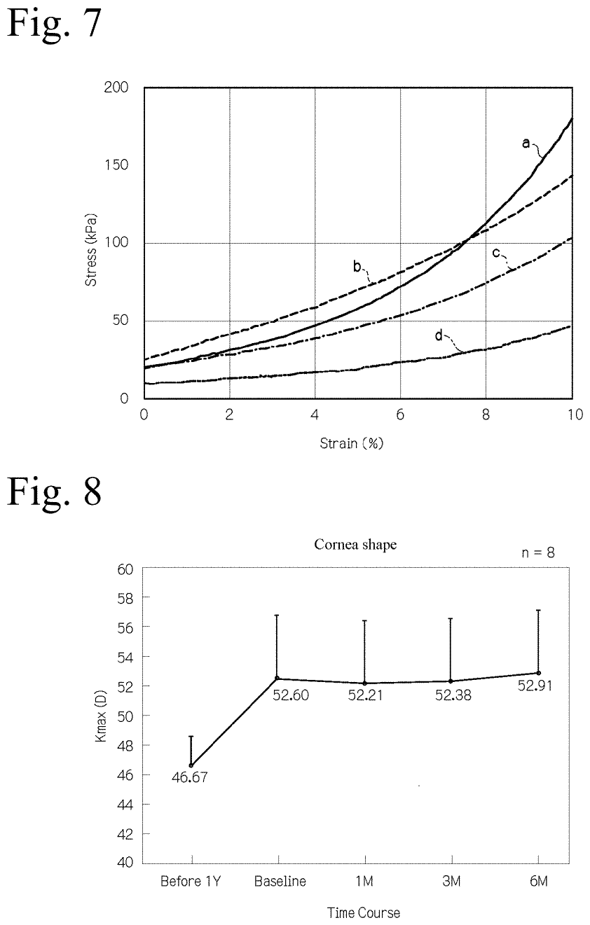

Experiment 4 was performed on a pig eye cornea. The results of the tensile tests are shown in FIG. 7. The experimental means were the same as in Experiment 1, except that the subject was a pig eye. As shown by reference sign a in FIG. 7, increases in the strength of the pig eye corneal tissues by the FAD eye drops and violet light irradiation were confirmed (KeraVio). Increases in the strength of the corneal tissue were confirmed by the conventional corneal crosslinking method (Riboflavin) by riboflavin eye drops shown by reference sign c of FIG. 7 and by the method (Ribo releasing CL) by sustained release from a riboflavin-containing soft contact lens and violet light irradiation shown by reference sign b of FIG. 7 as well. It should be noted that the reference sign d of FIG. 7 denotes the control.

### Experiment 5

In Experiment 5, a human cornea was targeted. Otherwise, similar to Experiment 1, FAD ophthalmic solution containing 0.53 mg/mL of FAD were used and violet light was irradiated. As the violet light, a light-emitting diode (LED) having a wavelength of 375 nm (manufactured by Nitride Semiconductors Co., Ltd., model name: Flashlight) was used, and the irradiance thereof was set to 310 μW/cm². This violet light was continuously irradiated for three hours per day, and eye drops were applied six times total at intervals of 30 minutes during the three hours. The total eye drop amount administered in six doses was 50 to 60 μg.

This was continuously performed for one month, three months, and six months, respectively. In all examples, epithelial ablation was not performed. After each period elapsed, a refractive power and an eyeglass-corrected visual acuity were measured. The refractive power was measured by using an anterior segment analyzer (product name: CASIA, manufactured by Tomey Corporation). Further, the eyeglass-corrected visual acuity was evaluated by the best spectacle-corrected visual acuity (BSCVA) using a visual acuity chart (Landolt ring).

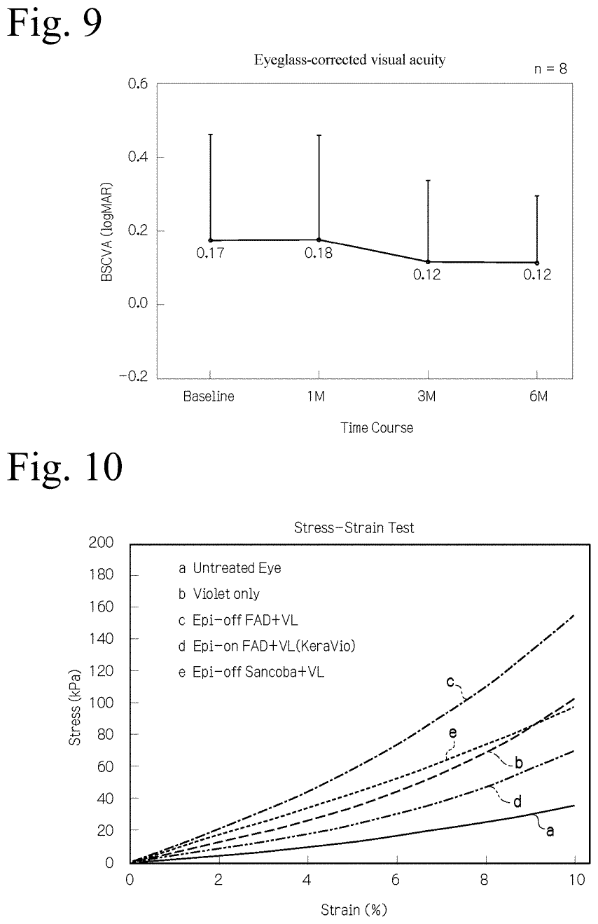

FIG. 8 is a graph (n=8) showing change over time of a maximum value of a refractive power (D) of a human cornea. FIG. 9 is a graph (n=8) showing change over time of BSCVA. From the results of FIG. 8 and FIG. 9, the maintenance of the corneal refractive power and the maintenance or improvement of the eyeglass-corrected visual acuity by FAD eye drops and violet light irradiation were confirmed. In each of the graphs, a line extending above the line graph indicates a range of standard deviation.

### Experiment 6

In Experiment 6, a pig eye cornea was targeted. With regard to the experimental conditions and experimental means, first, as the violet light, an LED having a wavelength of 375 nm (manufactured by Nitride Semiconductors Co., Ltd., model name: Flashlight) was used, and the irradiance thereof was set to 310 μW/cm². This violet light was continuously irradiated for 4.8 hours. Here, tensile tests were conducted under various conditions, such as a presence or an absence of corneal epithelium ablation and a change in the eye drops. The results of the tensile tests are shown in FIG. 10.

In FIG. 10, reference sign a denotes the result in a case where no treatment was carried out. Reference sign b denotes a case where the epithelium was not ablated (Epi-on) and only violet light was irradiated. Reference sign c denotes a case where epithelial ablation of the conventional method was performed (Epi-off), subsequently FAD eye drops (the same eye drop conditions as in Experiment 1) were applied, and violet light was irradiated (at irradiance of 310 μW/cm² for 4.8 hours continuously). Reference sign d denotes a case where FAD eye drops (the same eye drop conditions as in Experiment 1) were applied without performing epithelial ablation (Epi-on) , and violet light was irradiated (at irradiance of 310 μW/cm² for 4.8 hours continuously). Reference sign e denotes a case where epithelial ablation of the conventional method was performed (Epi-off), subsequently cyanocobalamin eye drops were applied, and violet light was irradiated (at irradiance of 310 μW/cm² for 4.8 hours continuously). It should be noted that, in the experiment of the reference sign e, the cyanocobalamin eye drops were applied six times total at 30-minute intervals during the three hours by using cyanocobalamin ophthalmic solution (trade name: Sancoba ophthalmic solution) containing 0.2 mg/mL of cyanocobalamin. The total eye drop amount was 36 to 60 ∥g.

As shown by reference sign din FIG. 10, it was confirmed that an increase in the strength of corneal tissue obtained by combining the administration of ophthalmic solution and the irradiation of violet light without performing epithelial ablation was to the same extent as that by the conventional invasive means. Further, as shown by reference sign b in FIG. 10, it was confirmed that an increase in the strength of corneal tissue obtained by simply the irradiation of violet light even without the administration of ophthalmic solution or epithelial ablation was to the same extent as that by the conventional means. It should be noted that, as shown by reference signs c and e in FIG. 10, although conventional invasive means were used, it was confirmed that Sancoba ophthalmic solution other than FAD ophthalmic solution (cyanocobalamin is conventionally used for asthenopia) as the eye drops can also be utilized as a medicine for improving corneal strength.

What is claimed is:

1. A non-invasive cornea and sclera strengthening device that irradiates violet light toward an eye administered with an administration agent used to strengthen a corneal tissue or a scleral tissue,
an irradiance of the violet light onto a surface of the eye being within a range of 0.1 to 1 mW/cm², and
a time of irradiation of the violet light onto the eye being within a range of one to five hours per day.

2. The non-invasive cornea and sclera strengthening device according to claim 1,
the non-invasive cornea and sclera strengthening device being a facially oriented-type device disposed so that the violet light is irradiated in a direction of the eye, and being one or two or more devices selected from facially oriented-type light source devices, such as eyeglasses with a light source, a liquid crystal display with a light source, a desk lamp, a handy light source, a table-top installed-type light source, and a personal computer/mobile terminal mounted-type light source.

3. The non-invasive cornea and sclera strengthening device according to claim 1 or 2, wherein
an administrating device of the administration agent is integrated with the facially oriented-type device.

4. The non-invasive cornea and sclera strengthening device according to any one of claims 1 to 3, wherein
the administration agent is administered to the eye by one or two or more methods selected from eye drops, eye ointment, sustained release from an eyeglass frame, and sustained release from a contact lens.

5. The non-invasive cornea and sclera strengthening device according to claim 4, wherein
the contact lens transmits violet light.

6. The non-invasive cornea and sclera strengthening device according to any one of claims 1 to 5, wherein
administration of the administration agent is carried out periodically or continuously.

7. The non-invasive cornea and sclera strengthening device according to any one of claims 1 to 6, wherein
the non-invasive cornea and sclera strengthening device is applied to keratoconus or other corneal diseases such as corneal ectasia, corneal infection, bullous keratopathy or the like, and ametropia.

8. A non-invasive cornea and sclera strengthening device that irradiates violet light toward an eye to strengthen a tissue of a cornea or a sclera,
an irradiance of the violet light onto a surface of the eye being within a range of 0.1 to 1 mW/cm², and
a time of irradiation of the violet light onto the eye being within a range of one to five hours per day.

9. A non-invasive cornea and sclera strengthening method, comprising the step of:
using a non-invasive cornea and sclera strengthening device capable of setting an irradiance of violet light onto a surface of an eye within a range of 0.1 to 1 mW/cm², and setting a time of irradiation of the violet light onto the eye within a range of one to five hours per day and, at a timing after administration of an administration agent used for strengthening a tissue of a cornea or a sclera to the eye, irradiates the violet light by the non-invasive cornea and sclera strengthening device.

10. A non-invasive cornea and sclera strengthening method, comprising the step of:
using a non-invasive cornea and sclera strengthening device capable of setting an irradiance of violet light onto a surface of an eye within a range of 0.1 to 1 mW/cm², and setting a time of irradiation of the violet light onto the eye within a range of one to five hours per day.
